(19) United States
(12) Patent Application Publication
(10) Pub. No.: US 2010/0330033 A1
(43) Pub. Date: Dec. 30, 2010

(54) Protein-carrier conjugates

(76) Inventors: Nian Wu, North Brunswick, NJ (US); Brian Charles Keller, Antioch, CA (US)

Correspondence Address: Lee Pederson, 712 East Main Street, Sleepy Eye, MN, 56085, US

(21) Appl. No.: 12/799,006

(22) Filed: Apr. 15, 2010

Related U.S. Application Data
(60) Provisional application No. 61/212,825, filed on Apr. 16, 2009.

(51) Int. Cl. A61K 38/21; C07K 17/08; A61K 38/28; A61P 3/10

(52) U.S. Cl. 424/85.4; 530/351; 530/303; 514/5.9

(57) ABSTRACT

The invention provides compositions and methods for covalent attachment of polymer and lipid carriers to therapeutic proteins to form carrier-protein conjugates having linkers between the carrier and protein portions of the conjugates. The linkers are selected to minimize steric effects. The linkers reduce the shielding effect of the carrier on the therapeutic protein and also allow better access for enzymatic or chemical cleavage of the carbamate bond. The linkers attach to the therapeutic protein via a carbamate bond and are either directly adjacent to the carbamate bond or are separated by a single carbon having a nitrogen side chain. Such linkers are solely comprised of carbon, sulfur and hydrogen and are between four and ten atoms (either C or S) in length.

### PRIORITY CLAIM

This application claims priority to U.S. provisional patent application No. 61/212,825 entitled “POLYMER LIPID PROTEIN CONJUGATES AND PREPARATION” and filed on Apr. 16, 2009.

### FIELD OF THE INVENTION

The present invention is related to protein-carrier conjugates, including protein-polymer conjugates and protein-lipid-polymer conjugates. In particular, the invention is related to conjugates having a novel linkage between the protein and at least a portion of the carrier portion of the conjugate.

### BACKGROUND OF THE INVENTION

Polyethylenglycol (PEG) is widely used as a water soluble carrier for polymer-drug and protein conjugates. PEG is undoubtedly the most studied and applied synthetic polymer in the biomedical field [R. Duncan, Nature Rev. Drug Discov. 2 (2003) 347-360]. As an uncharged, water-soluble, nontoxic, nonimmunogenic polymer, PEG is an ideal material for biomedical applications. Covalent attachment of PEG to biologically active compounds is often useful as a technique for alteration and control of biodistribution and pharmacokinetics, minimizing toxicity of these compounds [R. Duncan, and J. Kopecek, Adv. Polym. Sci. 57 (1984) 53-101]. PEG possesses several beneficial properties: very low toxicity [S Pang, J. Am. Coil. Toxicol, 12 (1993) 429-456], excellent solubility in aqueous solutions [G. M. Powell, Handbook of Water Soluble Gums and Resins, R. L. Davidson (Ed.), Ch. 18 (1980), MGraw-Hill, New York], and extremely low immunogenicity and antigenicity [S, Dreborg, Crit. Rev. Ther. Drug Carrier Syst., 6 (1990) 315-365]. The polymer is known to be non-biodegradable, yet it is readily excretable after administration into living organisms. In vitro study showed that its presence in aqueous solutions has shown no deleterious effect on protein conformation or activities of enzymes. PEG also exhibits excellent pharmacokinetic and biodistribution behavior. [T. Yamaoka, Y. Tabata, and Y. Ikada, J. Pharm. Sci., 83 (1994) 601-606].

In the early developmental stage of PEGylation, the attention has been focused on the amino groups, which are the most represented groups in proteins and are the most suitable conjugation sites. Amino groups are generally exposed in an aqueous environment or other solvent, and can be modified with a wide selection of chemical strategies. Several conjugation strategies are now available, such as alkylation, which maintains the positive charge of the starting amino group because a secondary amine is formed, or acylation, accompanied by loss of charge. [L. M. Graham, Adv. Drug Deliv. Rev. 55 (2003) 1293-1302; Y. Levy, et al., J. Pediatr. 113 (1988) 312-317; P. Bailon, et al., Bioconjug. Chem. 12 (2001) 195-202; Y. S. Wang, et al., Adv. Drug Deliv. Rev. 54 (2002) 547-570; O. B. Kinstler, et al., Pharm. Res. 13 (1996) 996-1002; S. S. Wong, Chemistry of protein conjugation and cross-linking, p. 13 (1991), CRC Press; P. Caliceti, et al., J. Bioact. Comp. Polym. 8 (1993) 41-50]

Esters with PEG have been utilized in chemical modifications of drugs. PEG esters which have an electron withdrawing substituent (alkoxy) in the a-position have proved to be especially effective linking groups in the design of prodrugs since the substituent aids in the rapid hydrolysis of the ester carbonyl bond, thus releasing alcohols in a continuous and effective manner. For instance, highly water soluble PEG-5000 esters of paclitaxel were synthesized and shown to function as prodrugs, i.e., breakdown occurred in a predictable fashion in vitro. [R. B. Greenwald, A. Pendri, D. Bolikal, C. W. Gilbert, Bioorg. Med. Chem. Lett. 4 (1994) 2465-2470]. Studies also showed that amino acid conjugates appeared to be the most useful, reducing toxicity while increasing efficacy for most of the anticancer drugs [A. Pendri, C. D. Conover, R. B. Greenwald, Anti-Cancer Drug Design, 13 (1998) 387-395; R. B. Greenwald, A. Pendri, C. D. Conover, C. Lee, Y. H., Choe, C. Gilbert, A. Martinez, J. Xia, D. Wu, M. Hsue, Bioorg. Med. Chem. 6 (1998) 551-562].

Study showed that dietary intake of long-chain omega-3 polyunsaturated fatty acids, eicosapentaenoic acid, and docosahexaenoic acid can affect numerous processes in the body, including cardiovascular, neurological and immune functions, as well as cancer [P D. Biondo, D. N. Brindley, M. B. Sawyer, C. J. Field, J Nutr Biochem. 19 (2008) 787-96]. Most significantly, lipids such as 1,2-diacylglycerol can activate protein enzymes [T J. Nelson, M K. Sun, J. Hongpaisan, D L. Alkon, Eur J Pharmacol. 585 (2008)76-87]. Monoglycerides or diglycerides are surface active molecules having both the hydrophobic and electrostatic components which mediates membrane trafficking and protein sorting in cells [L. Gelman, G. Zhou, L. Fajas, E. Raspé, J C. Fruchart, J. Auwerx, J Biol Chem. 274 (1999)7681-8; G N. Moll, W N. Konings, A J. Driessen, Antonie Van Leeuwenhoek. 76 (1999)185-98; JA. Corbin, J H. Evans, K E. Landgraf, J J. Falke, Biochemistry, 46 (2007) 4322-36].

### BRIEF SUMMARY OF THE INVENTION

The invention provides compositions and methods for covalent attachment of polymer and lipid carriers to therapeutic proteins to form carrier-protein conjugates having linkers between carrier and protein portions of the conjugates. The linkers are selected to minimize steric effects. The linkers reduce the shielding effect of the carrier on the therapeutic protein and also allow better access for enzymatic or chemical cleavage of the carbamate bond. The linkers attach to the therapeutic protein via a carbamate bond and are either directly adjacent to the carbamate bond or are separated by a single carbon having a nitrogen side chain. Such linkers are solely comprised of carbon, sulfur and hydrogen and are between four and ten atoms (either C or S) in length.

### BRIEF DESCRIPTION OF THE DRAWINGS

FIG. 1 depicts potential cleavage sites of αN-mPEG-αN-laurate-lysine-phenylalanine

FIG. 2 depicts stability profile of εN-mPEG-αN-laurate-lysine-phenylalanine

FIG. 3 depicts stability of laurate-lysine carbamade

FIG. 4 depicts Stability of Lysine-Phenylalanine Carbamade

### DETAILED DESCRIPTION OF THE INVENTION

Embodiments of the present invention are described herein in the context of protein-carrier conjugates having linkers to minimize the effects of steric hindrance. Those of ordinary skill in the art will realize that the following detailed description of the present invention is illustrative only and is not intended to be in any way limiting. Other embodiments of the present invention will readily suggest themselves to such skilled persons having the benefit of this disclosure.

In the interest of clarity, not all of the routine features of the implementations described herein are shown and described. It will, of course, be appreciated that in the development of any such actual implementation, numerous implementation-specific decisions must be made in order to achieve the developer's specific goals, such as compliance with application- and business-related constraints, and that these specific goals will vary from one implementation to another and from one developer to another. Moreover, it will be appreciated that such a development effort might be complex and time-consuming, but would nevertheless be a routine undertaking of engineering for those of ordinary skill in the art having the benefit of this disclosure.

PEG-protein conjugates can be used for modifying pharmacokinetic profiles due to increased blood half-life and decreased antigenicity [A. Kozlowski and J. M. Harris, J. Control. Release, 72 (2001) 217-224]. Due to water solvation of each ethylene oxide union of PEG polymers, a conjugated molecule acts as if were 5-10 times as large as a polymer of comparable molecular weight [A Kozlowski and J. M Harris, J. Control. Release, 72 (2001) 217-224] which can significantly extend its circulation time in the body since the clearance rate of a PEG-conjugate is inversely proportional to its molecular weight [T. Yamaoka, Y. Tabata and Y. Ikada, J. Pharm. Sci., 83 (1994) 601-606]. For instance, while a molecule with a mass of 20,000 or below will be eliminated primarily through kidney, larger molecules of PEG-conjugates are slowly cleared through both of liver and kidney [T. Yamaoka, Y. Tabata and Y. Ikada, J. Pharm. Sci. 83 (1994) 601-606].

As mentioned the above, conjugating polymers to therapeutic biological molecules such as insulin and interferon proteins has shown that both biologic activity and physical properties of can be significantly enhanced. However, such conjugation may have a significant impact on the bioactivity [A. Basu, K. Yang, M. Wang, S. Liu, R. Chintala, T. Palm, H. Zhao, P. Peng, D. Wu, Z. Zhang, J. Hua, M C. Hsieh, J. Zhou, G. Petti G, X. Li, A. Janjua, M. Mendez, J. Liu, C. Longley, Z. Zhang, M. Mehlig, V. Borowski, M. Viswanathan, D. Filpula., Bioconjug Chem. 17 (2006) 618-30].

For example, significant differences in the specific activities at different binding sites were observed for positional isomers of monopegylated interferon (PEG-IFN) alpha-2a, which was belivieved that the varying antiviral activities of the various PEG-IFN positional isomers may be due to the chemical attachment of the large PEG moiety at different sites to the IFN which lead to changes of the receptor-ligand interactions, specific for each isomer. In addition, the steric hindrance between the protein structure in the environment of individual lysines and the large size of the PEG tails may reduce the yields of the individual pegylated species [S. Foser, A. Schacher, K A. Weyer, D. Brugger, E. Dietel, S. Marti, T. Schreitmüller, Protein Expr Purif. 30 (2003) 78-87].

Recent studies reported that the highest residual activity was observed with the His-34 positional isomers and the lowest was observed with the Cys-1 positional isomers. The Lys positional isomers demonstrated intermediate activity, with a general order of Lys-134>Lys-83=Lys-131=Lys-121>Lys-31. The higher specific activity associated with the His-34 positional isomer suggests that this site may be favorable for pegylating IFN-αab molecules [MJ. Grace, S. Lee, S. Bradshaw, J. Chapman, J. Spond, S. Cox, M. Delorenzo, D. Brassard, D. Wylie, S. Cannon-Carlson, C. Cullen, S. Indelicato, M. Voloch, R. Bordens, J. Biol. Chem. 280 (2005) 6327-36].

It also reported from these studies that the more likely reason for higher activity with the His-34 isomer is that the site of pegylation may reduce the impact of steric hindrance from the PEG molecule, increased steric hindrance at the binding interface may also contribute to the decreased activity observed with increased PEG molecule size, either cooperatively with or independently of the receptor binding interaction. The authors suggested that the higher in vitro specific activity of 12-kDa PEG-IFN-2b relative to 40-kDa PEG-IFN-2a can be attributed to differences in the respective size of the PEG moiety and the distribution of positional isomers. Increasing the PEG moiety size significantly attenuated the in vitro antiviral activity of all pegylation sites studied [MJ. Grace, S. Lee, S. Bradshaw, J. Chapman, J. Spond, S. Cox, M. Delorenzo, D. Brassard, D. Wylie, S. Cannon-Carlson, C. Cullen, S. Indelicato, M. Voloch, R. Bordens, J Biol Chem. 280 (2005) 6327-36].

Studies also showed that due to the opposing effects of reduced receptor binding affinity and prolonged conjugate circulating lives, enhancing clinical potency is a greater challenge for PEGylation of small protein ligands [Y. Yamamoto, Y. Tsutsumi, Y. Yoshioka, T. Nishibata, K. Kobayashi, T. Okamoto, Y. Mukai, T. Shimizu, S, Nakagawa, S, Nagata, and T. Mayumi, Nat. Biotechnol. 21 (2003) 546-552]. A receptor binding site may constitute a large proportion of the ligand surface area; for example, 960 Å² of accessible surface is buried in each binding interface of the IFN-γ/IFN-γRα complex (M. R. Walter, W. T. Windsor, T. L. Nagabhushan, D. J. Lundell, C. A. Lunn, P. J. Zauodny, and S. K. Narula, Nature, 37 (1995) 230-235]. Such steric hindrance is especially significant for smaller proteins and peptides.

The present invention addresses these deficiencies by employing a linear linker or spacer to reduce two types of steric problems associated with protein-carrier conjugates. First, the spacer creates a separation between the carrier and the protein, thereby reducing shielding effects from the carrier on the active site of the protein. Second, the spacer allows greater access for enzymatic breakdown of the carbamate bond which connects the protein to the rest of the conjugate.

There are two basic variations of the invention. The first variation is demonstrated by Chemical Structure 1. In Chemical Structure 1, an amine of a therapeutic protein (P) is conjugated via a carbamate bond to a linker and a carrier group (R). In the present invention, the linker is defined as a central component without specified functional groups or bonding properties at each ends of the starting materials which are available for conjugating to a protein or a polymer carrier. The linker consists of a linear and saturated chain of atoms of C and/or S. Examples of such linkers include —CH2-CH2-CH2-CH2-, —S—CH2-S—CH2-, and the like. Because the linkers do not have side chains and do not readily form hydrogen bonds in aqueous solutions, they effectively take up less volume than a comparable length of hydrophilic polymer such as PEG. Linkers between two and ten atoms of C and/or S are generally useful, although a minimum of four atoms is best. Longer linkers may introduce solubility problems, and therefore having between 4 and 6 atoms are preferable. Linkers having 4 atoms are most preferred.

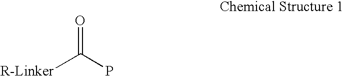

The second variation is demonstrated by Chemical Structure 2. In Chemical Structure 2, an amine of a therapeutic protein (P) is also conjugated via a carbamate bond to a linker and a carrier group (R1). As in Chemical Structure 1, the linker consists of a linear and saturated chain of between four and ten atoms of C and/or S. The difference between the variations is that this one includes an extra carbon atom between the carbamate bond and the linker, where the extra carbon atom has an attached nitrogen, which in turn may be attached to a second carrier group (R2). Alternatively, the attached nitrogen may be left as a simple amine group. Because of the flexibility of this portion of the conjugate, the steric advantages are largely maintained, with the added advantage of increases ease of synthesis and expanded carrier design. In this variation, R1 and R2 may collectively be referred to as the carrier portion of the conjugate.

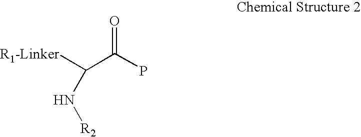

In both variations, the carrier group may include additional connecting elements, as shown below in various embodiments of the inventions. The carrier group or carrier portion includes at least one non-antigenic polymer, typically polyethylene glycol (PEG). The PEG may be branched or linear and each PEG may have a molecular weight between about 400 and 60,000 Daltons. The carrier group or carrier portion may also include lipids or fatty acids to improve cell permeation and transportation. Such lipids and fatty acids may be incorporated in a variety of ways, as exemplified in this disclosure.

The carbamate bond is preferable to an amide bond when coupling to protein amine groups, as the carbamate bond is more labile.

The linkers of the present invention my be incorporates into a wide variety of protein-carrier conjugates. Polymers, lipids, and proteins can be combined in numerous ways depending on the aims of the formulator. In addition to PEG, polymers that may be used include polyvinylpyrrolidine, polymethoxazoline, polyethyloxazoline, polyhydroxypropyl methacrilide, polymethacrylamide, polydimethacrylamide, polyacetic acid, polyglycolic acid, derivitized celluloses, as well as co-polymers and block co-polymers of the above. The polymers may be branched or linear, monodisperse or heterodisperse. Termini of the polymers may be varied, though mPEG is a preferred embodiment. Lipids may be selected from those shown in Tables 1 and 2, as well as others. Possible proteins for incorporation include Interferons (IFNs), Interleukins (ILs), Tumour Necrosis Factors (TNFs), Colony Stimulating Factors (CSFs), Erythropoietin (Epoetin/EPO) and Thymopoietins or recombinant human Growth Hormone (rhGH) or monoclonal antibodies including Infliximab and Cetuximabng or Peptide-based drug molecules including Insulin and Enfuvirtide, etc.

TABLE 1
Saturated lipids for use in the invention:

| | | | | Melting |
|---|---|---|---|---|
| common | | | | point |
| name | IUPAC name | Chemical structure | Abbr. | (° C.) |
| Caprylic | Octanoic acid | CH₃(CH₂)₆COOH | C8:0 | 16-17 |
| Capric | Decanoic acid | CH₃(CH₂)₈COOH | C10:0 | 31 |
| Lauric | Dodecanoic acid | CH₃(CH₂)₁₀COOH | C12:0 | 44-46 |
| Myristic | Tetradecanoic acid | CH₃(CH₂)₁₂COOH | C14:0 | 58.8 |
| Palmitic | Hexadecanoic acid | CH₃(CH₂)₁₄COOH | C16:0 | 63-64 |
| Stearic | Octadecanoic acid | CH₃(CH₂)₁₆COOH | C18:0 | 69.9 |
| Arachidic | Eicosanoic acid | CH₃(CH₂)₁₈COOH | C20:0 | 75.5 |
| Behenic | Docosanoic acid | CH₃(CH₂)₂₀COOH | C22:0 | 74-78 |

TABLE 2
Unsaturated lipids

| | | Δ^{x} | |
|---|---|---|---|
| | | Location of | # carbon/ |
| Name | Chemical structure | double bond | double bonds |
| Myristoleic acid | CH₃(CH₂)₃CH═CH(CH₂)₇COOH | cis-Δ⁹ | 14:1 |
| Palmitoleic acid | CH₃(CH₂)₅CH═CH(CH₂)₇COOH | cis-Δ⁹ | 16:1 |
| Oleic acid | CH₃(CH₂)₇CH═CH(CH₂)₇COOH | cis-Δ⁹ | 18:1 |
| Linoleic acid | CH₃(CH₂)₄CH═CHCH₂CH═CH(CH₂)₇COOH | cis,cis-Δ⁹,Δ¹² | 18:2 |
| α-Linolenic acid | CH₃CH₂CH═CHCH₂CH═CHCH₂CH═CH(CH₂)₇COOH | cis,cis,cis- | 18:3 |
| | | Δ⁹,Δ¹²,Δ¹⁵ | |
| Arachidonic acid | CH₃(CH₂)₄CH═CHCH₂CH═CHCH₂CH═CHCH₂CH═CH(CH₂)₃COOH | cis,cis,cis,cis- | 20:4 |
| | | Δ⁵Δ⁸,Δ¹¹,Δ¹⁴ | |
| Erucic acid | CH₃(CH₂)₇CH═CH(CH₂)₁₁COOH | cis-Δ¹³ | 22:1 |

In the previous publications, individual mono-polymer-interferon conjugates are defined as positional isomers, depending upon which amino acid residue is covalently attached to the polymer (WO9513090, U.S. Pat. No. 5,738,846, U.S. Pat. No. 6,042,822). In the present invention, the impact from positional isomers may be minimized by the linker.

The linkers themselves may be varied, and starting materials may be chosen to optimize molecular design and ease of synthesis. The linker or spacer is between 2-10 atoms (C or S) long. More preferably, the linker is 4-6 atoms long. Most preferably, the spacer is 4 atoms long. Convenient starting materials for linkers in the first variation of the invention (Chemical Structure 1) include those shown in Table 3.

TABLE 3
Linear Amino Carboxylic Acids

| Systematic (IUPAC) | | |
|---|---|---|
| name | Common name | Chemical Structure |
| 5-Aminopentanoic acid | 5-Amino-n-valeric acid | 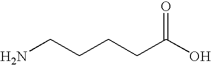 |
| 6-Aminohexanoic acid | 6-Amino-n-caproic acid | 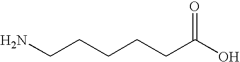 |
| 7-Aminoheptanoic acid | 7-Aminooenanthic acid | 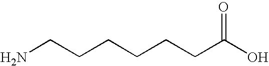 |
| 8-Aminooctanoic acid | 8-amino-n-caprylic acid | 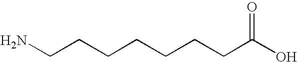 |
| 9-Aminononanoic acid | 9-Aminopelargonic acid | 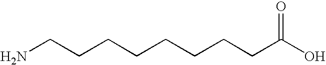 |
| 10-Aminodecanoic acid | 10-Aminocapric acid | 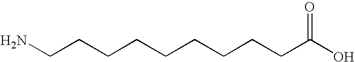 |
| 11-Aminoundecanoic acid | 11-Aminoundecanoic acid | 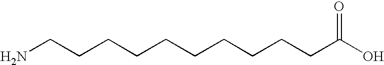 |

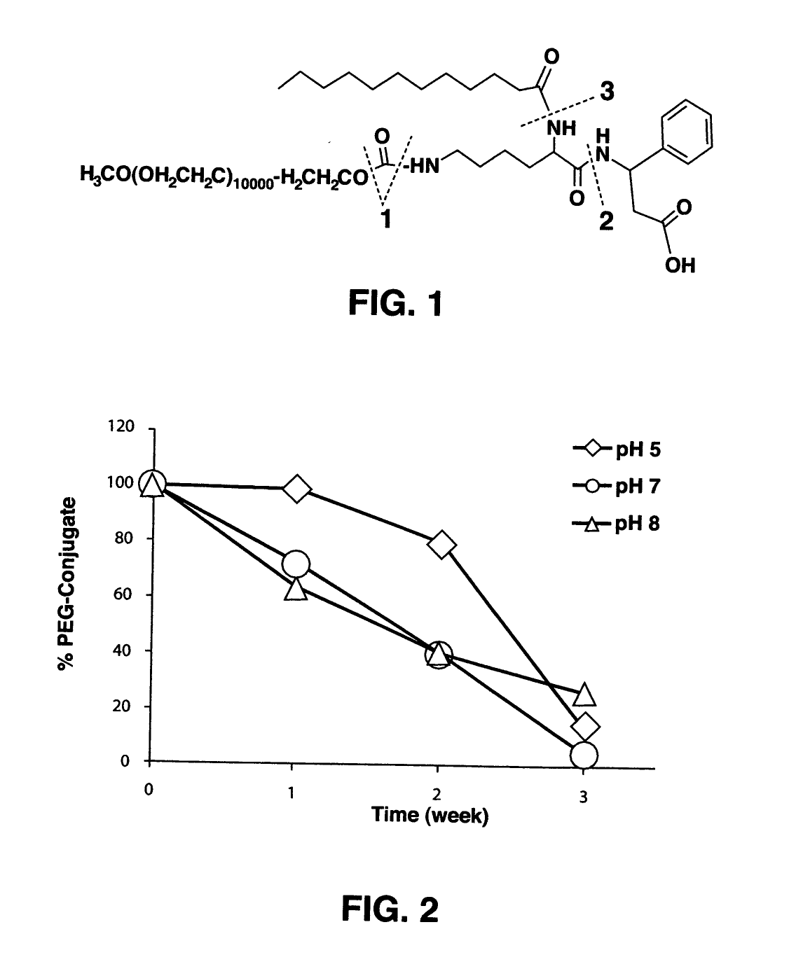

When synthesizing conjugates of the second variation of the invention (Chemical Structure 2) lysine is a convenient starting material to comprise the linker. Lysine provides a linear “space” of accessible surface area approximately 314 A² (FIG. 1) or 8.6 Å in length. Lysine's other advantages are its ease in conjugating to protein or peptide amines via carbamate bond while having easily modifiable amine groups to attach carrier portions.

Some examples of variations of the invention are shown in Table 4. In Table 4, R1 and R2 can be the same or different from selected diglycerides or fatty acids (Tables 1 and 2) and mono-polymer such as PEG.

TABLE 4
Structural variations of the invention

| Linear amino carboxylic acid based linker | Alpha Amino acid based linker |
|---|---|
| 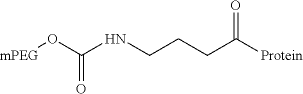 | 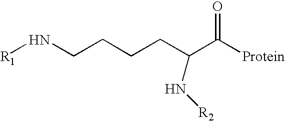 |
| 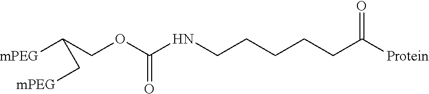 | |
| 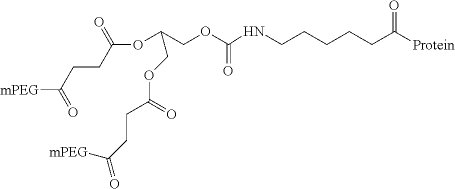 | 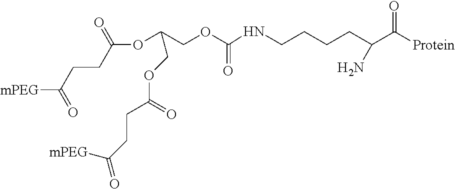 |
| 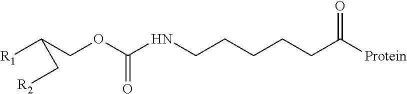 | |

Generally, the first synthesis steps entail attaching the carrier group or carrier portion to the starting material comprising the linker. The protein-polymer conjugates are then prepared in a solution by reacting protein or peptide with appropriate amounts of carrier-linker conjugate.

While various activation agents are suitable for the protein conjugation in the present invention, the more preferable conjugation agents are N-succinimidyl chlorormate or Disuccinimidylcarbonate or Biotinamidocaproate N-hydroxysuccinimide ester or Biotinamidohexanol N-hydroxysuccinimide carbonate or Biotinamidohexylamine N-hydroxylsuccinimide carbamate or 1-(2,4-dinitrophenyl)-aminohexanol N-hydroxysuccinimide carbonate or 1-(2,4-dinitrophenyl)-aminohexanol N-hydroxysuccinimide carbamate. The most preferable activation agents are N-succinimidyl chlorormate or Disuccinimidylcarbonate or Biotinamidocaproate N-hydroxysuccinimide ester or Biotinamidohexanol N-hydroxysuccinimide carbonate or Biotinamidohexylamine N-hydroxylsuccinimide carbamate.

In one aspect the invention is a conjugate of a therapeutic protein, the conjugate comprising the therapeutic protein; a carrier group including a non-antigenic hydrophilic polymer; a linker disposed between the protein and the carrier group, said linker attached to the therapeutic protein via a carbamate bond and located directly adjacent to the carbamate bond, and said linker comprising a linear and saturated chain of between four and ten atoms of C and/or S. The carrier group may comprise a single liner polyethyleneglycol (PEG) chain. The carrier group may comprise a branched polyethyleneglycol (PEG) chain. The carrier group may comprise a PEG chain conjugated to a lipid or fatty acid.

In another aspect, the invention is a conjugate of a therapeutic protein, the conjugate represented by the formula:

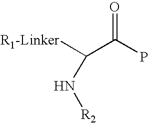

where P is a therapeutic protein; where R1 is selected from the group comprising a non-antigenic hydrophilic polymer, a lipid or a fatty acid; where R2 is selected from the group comprising a non-antigenic hydrophilic polymer, a lipid, a fatty acid, or two hydrogen atoms; and a the linker comprises a linear and saturated chain of between four and ten atoms of C and/or S. R1 and R2 may comprise two polyethyleneglycol (PEG) chains. R1 and R2 may comprises a polyethyleneglycol (PEG) chain and a lipid moiety. The linker may be derived from lysine.

In another aspect, the invention is a method for preparing a conjugate of a therapeutic protein, the method comprising: (step 1) conjugating one or more carrier groups to a linker, where the carrier groups are selected from the group comprising a non-antigenic hydrophilic polymer, a lipid or a fatty acid, and where the linker is a linear and saturated chain of between four and ten atoms of C and/or S; and (step 2) conjugating the product of step 1 to a therapeutic protein or peptide via a carbamate bond.

In another aspect, the invention is a method of treating a patient with a therapeutic protein, where the therapeutic protein is formulated as a conjugate according to paragraphs [038] and [039].

While embodiments and applications of this invention have been shown and described, it would be apparent to those skilled in the art having the benefit of this disclosure that many more modifications than mentioned above are possible without departing from the inventive concepts herein. The invention, therefore, is not to be restricted except in the spirit of the appended claims.

### Example 1

### Preparation of 1,2-di-mPEG glycerol

A 1,2-di-mPEG glycerol was prepared by the following steps (Chemical reaction scheme 1) and the molecular weight of mPEG is ranging from 400 to 20,000.

### Step 1: 1,2-Isopropylidenene-rac-glycerol-3-β,β,β-trichloroethylcarbonate (PRODUCT I)

A solution of 200 g (0.943 moles) of 2,2,2(β,β,β)-Trichloroethoxycarbonyl chloride in 100 mL of CHCl₃ (fresh distilled from P205) was added drop-wise to an ice-cold mixture of 124.6 g (0.942 moles) of DL-1,2-Isopropylidene-rac-glycerol, 50 mL of dry pyridine and 100 mL of CHCl₃. The solution was stirred at room temperature for 18 hrs, diluted with Et₂O (800 mL), and wash with successively with dilute HCl, H₂O, 5% NaHCO₃ and H₂O. The organic extract was dried with Na₂SO₄, then concentrated and distilled. To give a >85% of the colorless syrup: bp: 140-145° C. (0.25 mm Hg).

### Step 2: β,β,β-trichloroethyl carbonate glycerol (PRODUCT II)

Method (A) Hydrolysis with HCl: a mixture of 126 g (0.41 moles) of PRODUCT 1,150 mL Et₂O, 40 mL of MeOH, and 40 mL of 3NHCl was stirred over night (>12 hours) at room temperature. The solvents were evaporated at 40° C. at H₂O aspirator pressure, the residue was extracted with EtOAc, and the organic phase was washed with brine 5 times. After being dried with Na₂SO₄, the solvent was evaporated and the residue was azeotroped several times with C₆H₆ at 40° C. The yield was quantitative (>90%).

### Step 3: β,β,β-trichloroethyl carbonate di-mPEG glycerol (PRODUCT III)

100 g of PRODUCT II (0.325 moles), 0.66 moles mPEG carbonate, 148 g of DCC (0.715 moles) and a catalytic amount of DMAP (8.74 g, 0.0715 moles) in anhydrous CH₂Cl₂ (400 mL) was stirred at 25° C. for 12 h under Ar/or N₂, after which the N,N′-dicyclohexylurea salts were precipitated and removed by filtration. The filtrates were evaporated under reduced pressure

### Step 4: DL-1,2-di-mPEG-rac-glycerol

0.374 moles of PRODUCT III was dissolved in a mixture of HOAc (375 mL) and Et₂O (250 mL), and cooled in an ice batch. 315 g of active Zinc is added and the suspension was stirred at 20-25° C. for 2-3 hours or until the reaction was completed. After dilute with 300 mL of (4/1, v/v), the inorganic reagents were filtered and the filter cake was washed with additional Et₂O—CHCl₃ solvents. The filtrate was washed with H₂O three times, 5% NaHO₃, and brine. After being dried (on Na₂SO₄), the solvent was evaporated at 30° C.

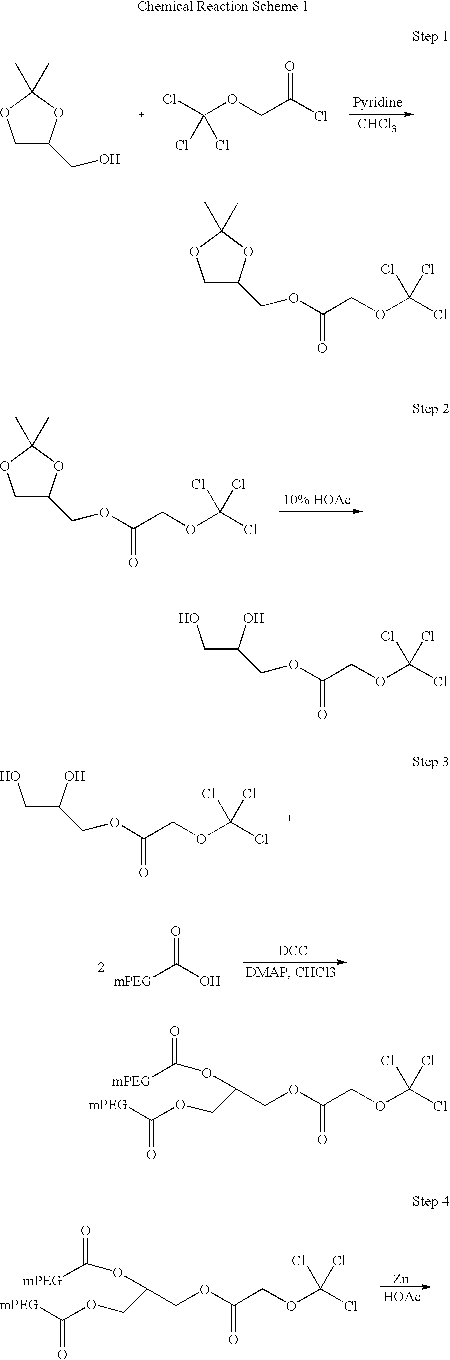

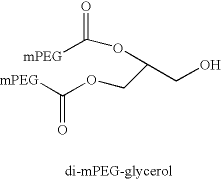

### Example 2

### Preparation N-hydroxysuccinimide ester of 1,2-di-mPEG-3-glycerol

0.1 moles of 1,2-di-mPEG-3-glycerol was added in 250 mL of dried dioxane and warmed up until completely dissolved. Gradually added 100 mL dry tetrahydrofuran solution of 0.6 moles of N-succinimidyl chlorormate and 100 mL dry tetrahydrofuran solution of 0.6 moles of 4-(dimethylamino)pyridine. Let reacted for 3 hours under constantly stirring. Filtered out the white precipitate of 4-(dimethylamino)pyridine HCl and the supernatant was collected. Added diethylether to the supernatant until no further precipitate was observed and dried the product and stored at −20° C. (see Chemical Structure 3).

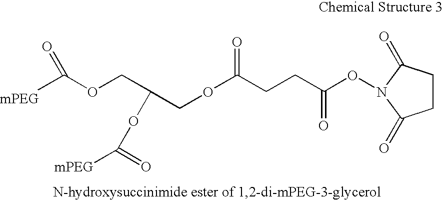

### Example 3

### Preparation of εN-1,2-di-mPEG-3-glycerol-lysine

0.1 moles of N_{α}-(tert-butoxycarbonyl)-L-lysine and 0.11 moles of N-hydroxysuccinimide ester of 1,2-di-mPEG-3-glycerol were dissolved in 160 mL of 0.1 M sodium carbonate (pH 9.5). The reaction mixture was stirred at 25° C. for 12 hr and diluted with water. The precipitate is collected via filtration and dried under vacuo (Chemical Structure 4).

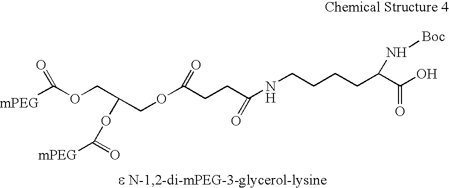

### Example 4

### Preparation of εN-1,2-di-mPEG-3-glycerol-N-hydroxysuccinimidyl-lysine

0.1 moles of starting material from Example 3 was dissolved in 250 mL of dried dioxane and warmed up until completely dissolved. Gradually added 100 mL dry tetrahydrofuran solution of 0.6 moles of N-succinimidyl chlorormate and 100 mL dry tetrahydrofuran solution of 0.6 moles of 4-(dimethylamino)pyridine. Let reacted for 3 hours under constantly stirring. Filtered out the white precipitate of 4-(dimethylamino)pyridine HCl and the supernatant was collected. Added diethylether to the supernatant until no further precipitate was observed and dried vacuo. The product was redissolved in 150 mL of CH₂Cl₂ and 40 mL of triethylamine was added and mix for 30 minutes to remove the butyl-protecting group on the alpha amine of lysine. Solvent was removed under vacuo and the crude product was further eluted in a silica gel column using a mobile phase consisting of chloroform, methanol and acetic acid (100:2:0.01) and major peak monitored at 210 nm was collected and dried. The final product was stored at −20° C. (Chemical Structure 5).

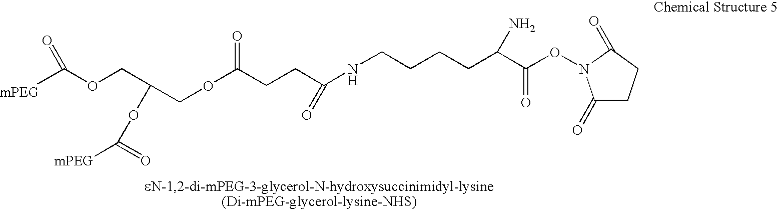

### Example 6

### Coupling Di-mPEG-glycerol-lysine-NHS to Proteins

PEGylation of target proteins was performed by adding di-mPEG₁₀₀₀-glycerol-lysine-NHS to a protein solution. For example, recombinant Interferon-alph-2b human (IFN α-2b, US Biological, Swampscott, Mass.) was dissolved, at a concentration of 1-10 mg/ml in 0.1 M phosphate buffer, pH 7.5. Di-mPEG₁₀₀₀₀-glycerol-lysine-NHS was added at a molar ratio of IFN:PEG=1:5 to 6 and reacted over night at 4° C. under constant stirring. The reaction was stopped by adjusting the pH of the solution to 4.5 with phosphoric acid. The resulting product was purified and excess PEG was removed by a sulfopropyl-sepharose cation-exchange chromatography using a salt gradient elution from 0 to 0.5 M NaCl in 25 mM Sodium citrate buffer (pH 5.0). The resulting product was stored at 4° C. (Chemical Structure 6).

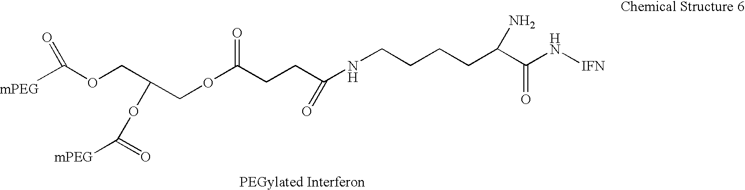

Using different starting materials and polymers, similar conjugates can be made following the steps described above Examples from 1 to 7 as listed in Tables 5 and 6.

TABLE 5
Samples of Polymer-Protein Conjugates

| Name | Structure |
|---|---|
| εN-1,2-di-mPEG-3- glycerol-αN-oleoyl- lysine-interferon | 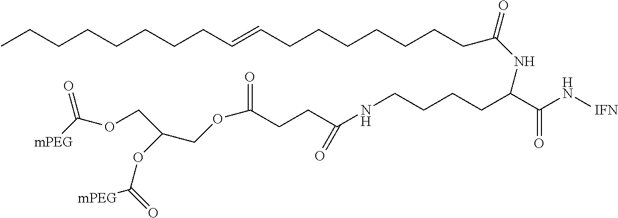 |
| εN-1,2-di-PEG-oleate-3- glycerol-lysine-interferon | 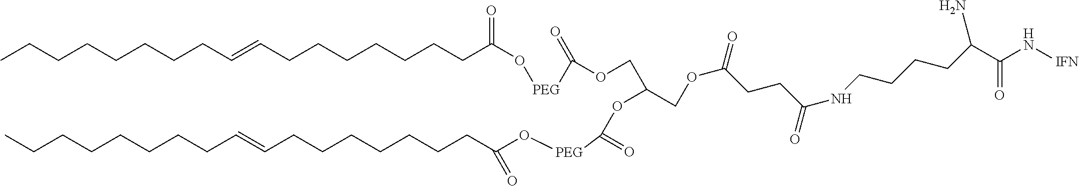 |
| εN-1,-oleoyl-2-mPEG-3- glycerol-lysine-interferon | 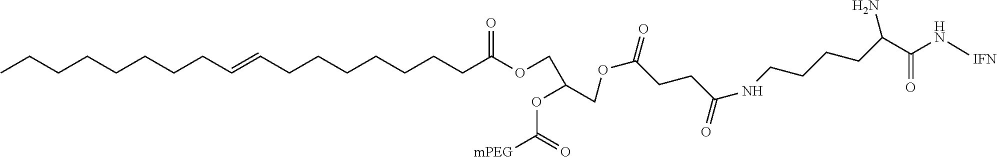 |
| εN-1,2-di-PEG-oleate-3- glycerol-αN-oleoyl- lysine-interferon | 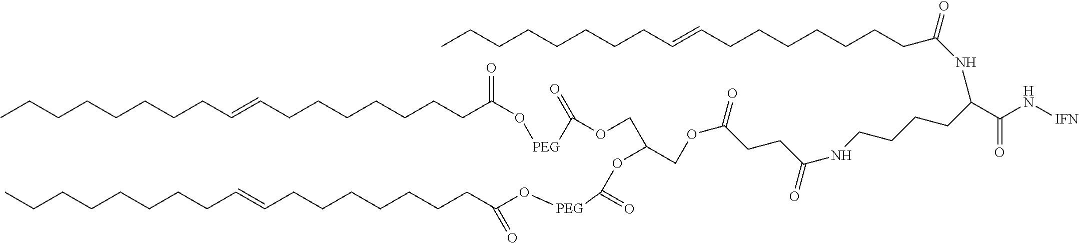 |
| εN-mPEG-lysine- interferon | 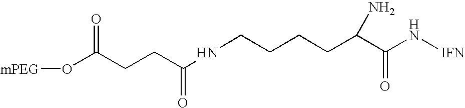 |
| εN-PEG-oleate-lysine- interferon | 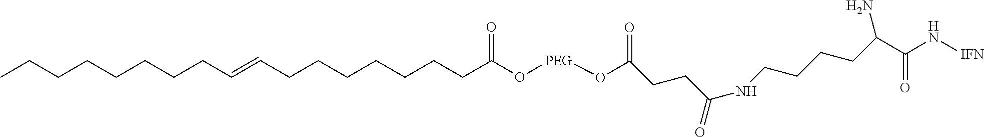 |
| εN-mPEG-αN-oleoyl- lysine-interferon | 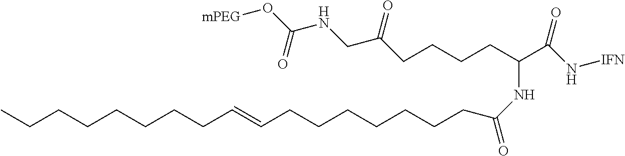 |
| εN-PEG-oleate-αN- oleoyl-lysine-interferon | 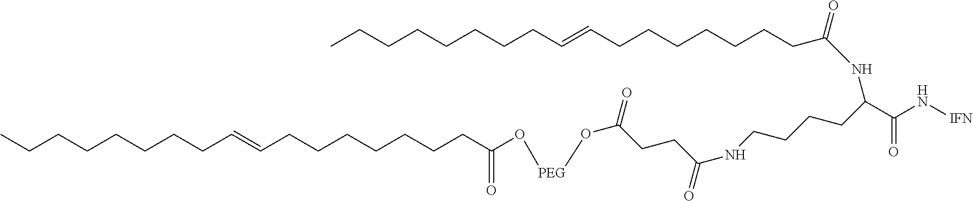 |
| di-mPEG-lysine- interferon | 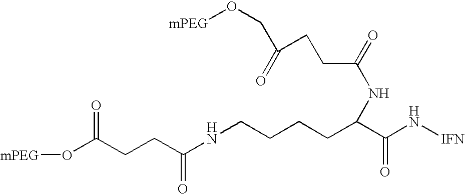 |
| di-PEG-oleate-lysine- interferon | 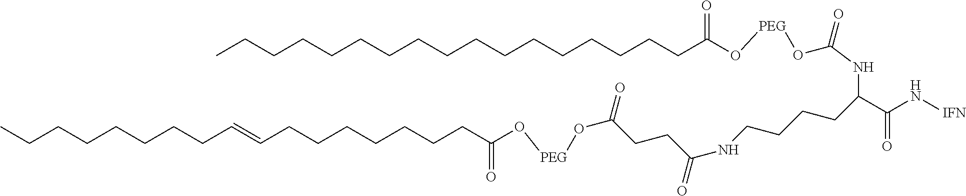 |
| εN-mPEG- aminohexanamide (AHA)-interferon | 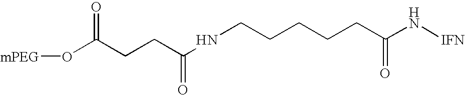 |
| εN-PEG-oleate-AHA- interferon | 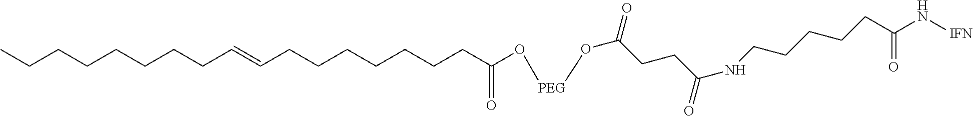 |
| εN-1,2-di-mPEG-3- glycerol-AHA-interferon | 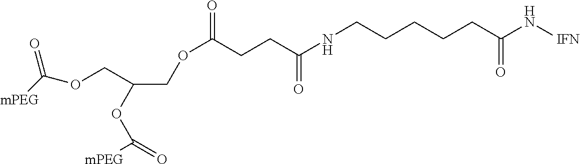 |
| εN-1,2-di-PEG-oleate-3- glycerol-hexanamide- interferon | 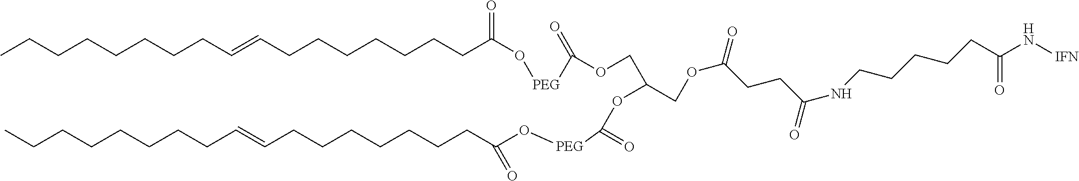 |
| εN-1-oleoyl-2-mPEG-3- glycerol-hexanamide- interferon | 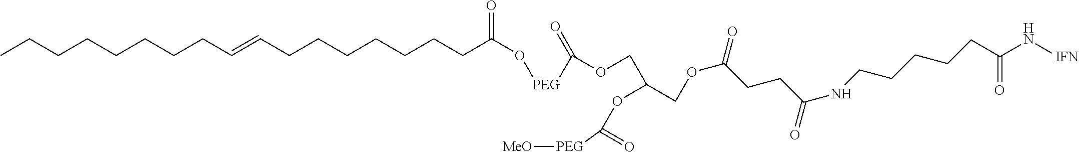 |

TABLE 6
Samples of Polymer-Peptide Conjugates

| Name | Structure |
|---|---|
| εN-1,2-di-mPEG-3-glycerol- lysine-insulin | 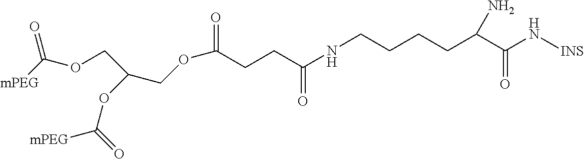 |
| εN-1,2-di-mPEG-3-glycerol- αN-oleoyl-lysine-insulin | 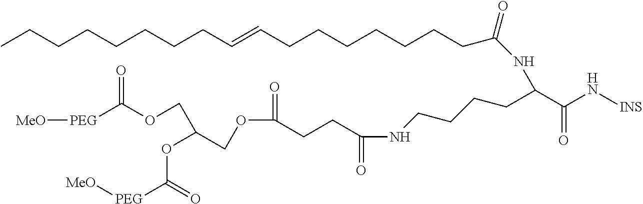 |
| εN-1,2-di-PEG-oleate-3- glycerol-lysine-insulin | 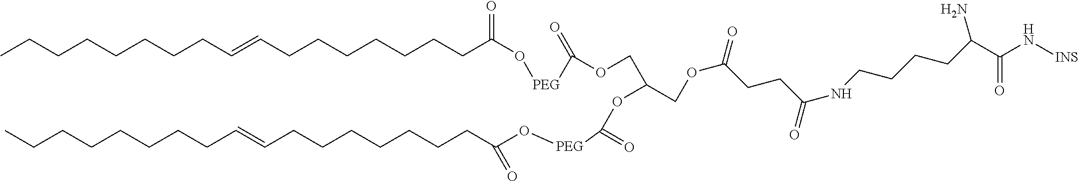 |
| εN-1, -oleoyl-2-mPEG-3- glycerol-lysine-insulin | 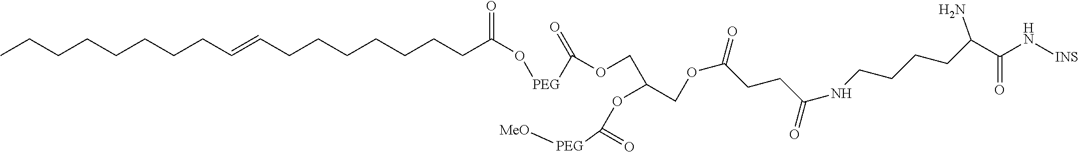 |
| εN-1,2-di-PEG-oleate-3- glycerol-αN-oleoyl-lysine- insulin | 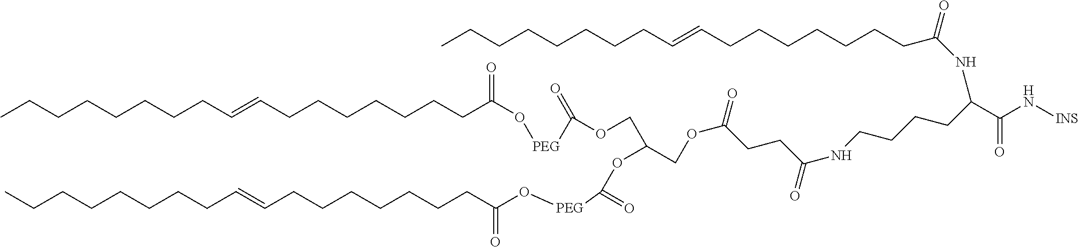 |
| εN-mPEG-lysine-insulin | 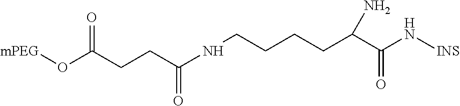 |
| εN-PEG-oleate-lysine-insulin | 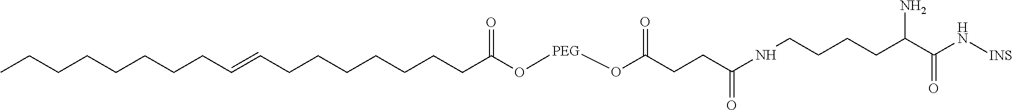 |
| εN-mPEG-αN-oleoyl-lysine- insulin | 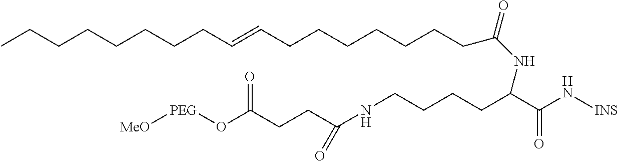 |
| εN-PEG-oleate-αN-oleoyl- lysine-insulin | 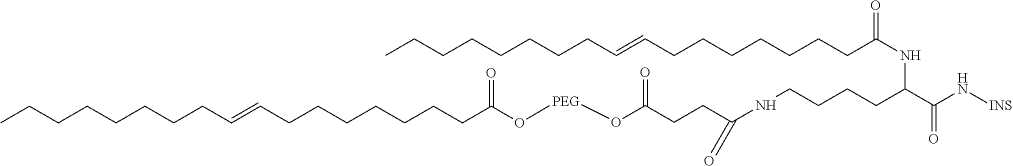 |
| di-mPEG-lysine-insulin | 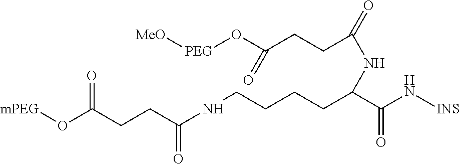 |
| di-PEG-oleate-lysine-insulin | 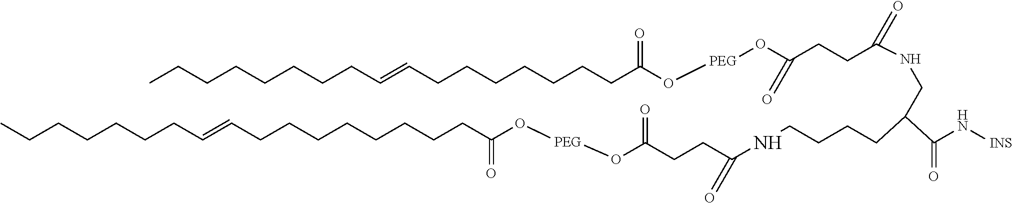 |
| εN-mPEG-AHA-insulin | 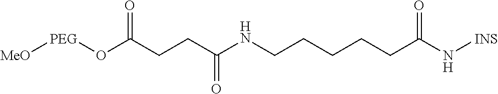 |
| εN-PEG-oleate-AHA-insulin | 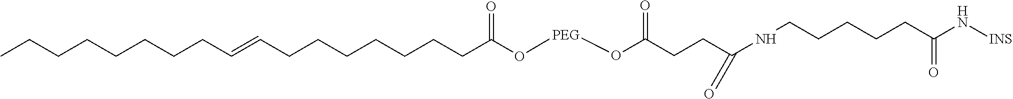 |
| εN-1,2-di-mPEG-3-glycerol- AHA-insulin | 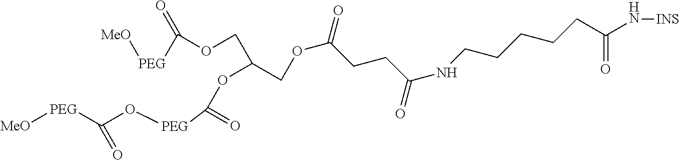 |
| εN-1,2-di-PEG-oleate-3- glycerol-hexanamide-insulin | 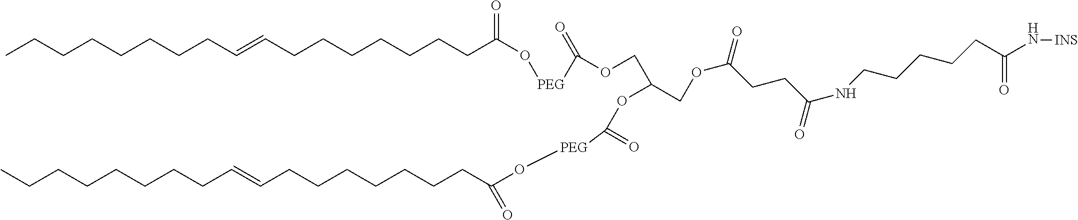 |
| εN-1-oleoyl-2-mPEG-3- glycerol-hexanamide-insulin | 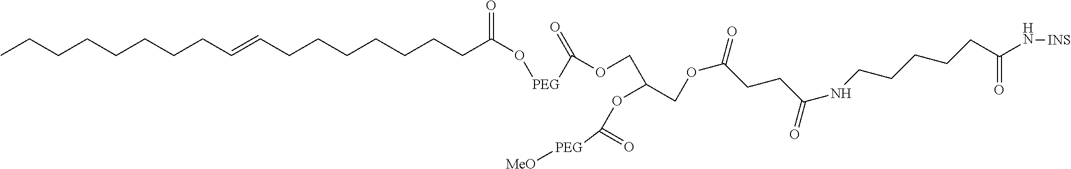 |

### Example 7

### Preparation of εn-mPEG-αN-Laurate-Lysine-Phenylalanine

20 mmoles of Boc-Lys-Phe and 22 moles of Lauric acid N-hydroxysuccinimide ester (Sigma-Aldrich) were dissolved in 50 mL of dimethylformamide and added 1.5 mL of triethylamine (TEA). The reaction mixture was stirred at 25° C. for 0.5 hr, added another 5 mL of TEA and stirred for another 30 minutes to remove the butyl-protecting group. The reaction was terminated by adding 100 mL of cool water. The precipitate was collected via filtration and dried under vacuo. 10 mM of the resulted product was re-dissolved in 0.1M Sodium Tetraborate (pH 9) under vigorous mixing, added mPEG₁₀₀₀₀-succinimidyl ester (Fisher Scientific, Pittsburgh, Pa.) as a solid to a targeted concentration ˜3 mM in the solution (αN-Laurate-Lysine-Phenylalanine was in a 300% molar excess). Maintained mild agitation when the activated PEG is fully dissolved. The reaction was complete in approximately 30 minutes (Chemical Structure 7). The PEG-conjugates was further purified and separated from mPEG-NHS, and from unreacted linker, by a gel filtration chromatography (Supderdex 200 HR 10/30, 24 mL bed volume).

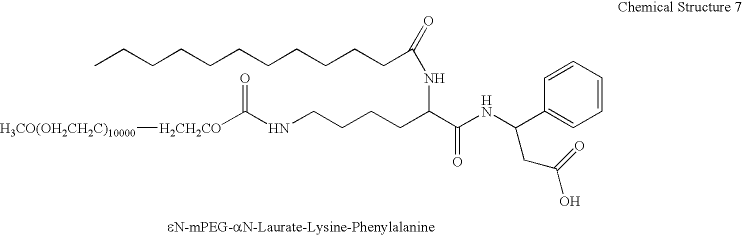

### Example 8

### Stability Study of εN-mPEG-αN-Laurate-Lysine-Phenylalanine

In order to demonstrate the stability of polyethylene glycol conjugates, εN-mPEG-αN-Laurate-Lysine-Phenylalanine was used as the model molecule and was treat with base or acid to examine the dissociation of the conjugate, the potential cleavage sites are showed as in FIG. 1.

Formation of αN-Laurate-Lysine-Phenylalanine is a result of the depegylation of εN-mPEG conjugate. Since the carbamide bond between a linker and IFN (2) is more stable than the carbamate bond (1) between mPEG and the linker, thus the cleavage of the carbamate bond is rate-limiting for releasing free IFN in vivo. The purpose of this stability study with the model molecule is used to demonstrate the prediction.

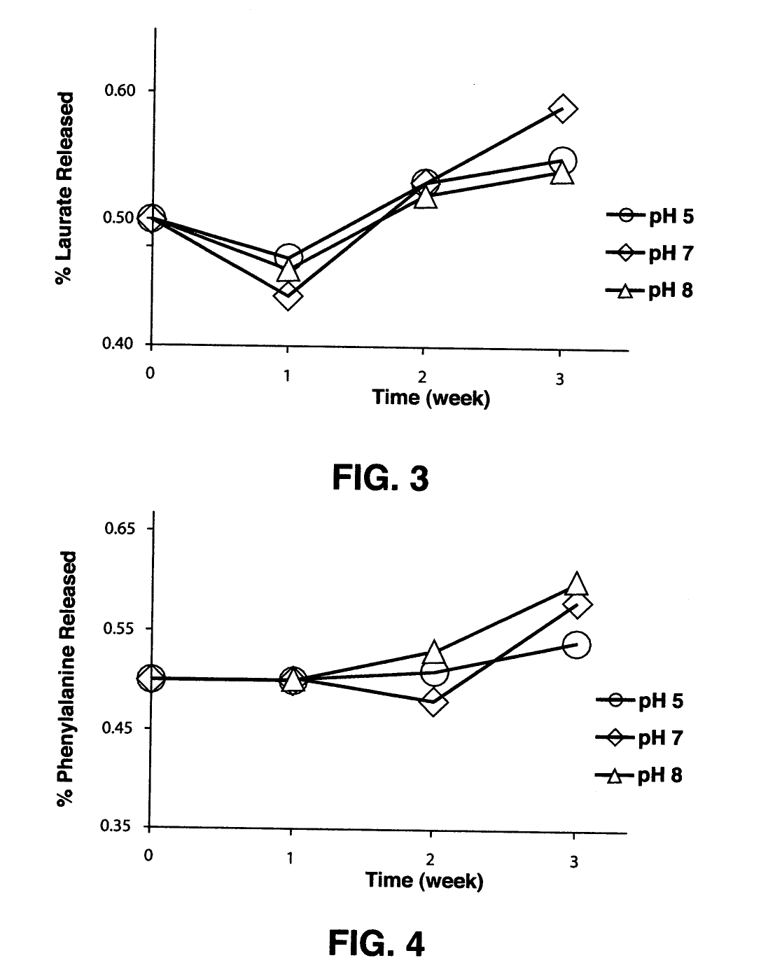

The rate of cleavage of εN-mPEG₁₀₀₀₀-αN-Laurate-Lysine-Phenylalanine was studied by tracking the release of αN-Laurate-Lysine-Phenylalanine. 0.5 mM of purified εN-mPEG₁₀₀₀₀-αN-Laurate-Lysine-Phenylalanine was tested for its stability by hydroxylamine treatment at neutral pH and incubation in solution at different pHs at 37° C. for up to 3 weeks. The cleavages of the conjugate from the stability samples were separated from εN-mPEG₁₀₀₀₀-αN-Laurate-Lysine-Phenylalanine by Amicon Ultra-15 Centrifugal Filter Devices with a molecular weight cut off of 5000 (Millipore, Billerica, Mass.), The concentrations of αN-Laurate-Lysine-Phenylalanine (m/z 476 (M+H)⁺), Phenylalanine (m/z 166, (M+H)⁺) and Lauric acid (m/z 201, (M+H)⁺) were assayed by a LC-MS method. The stability of εN-mPEG₁₀₀₀₀-αN-Laurate-Lysine-Phenylalanine in solution at different pHs is shown in FIG. 3. Based on the releasing rate of αN-Laurate-Lysine-Phenylalanine, the PEG-conjugate was relatively stable, with a specific hydrolysis rate of less than 3% per day across the pH range from 5.0 to 8.0 (Table 7). Both of the carbamide bonds of αN-Laurate-(3)-Lysine-(2)-Phenylalanine were very stable under the stress conditions for up to 3 weeks (FIGS. 4 and 5), the cleavages of carbamade bonds were remained virtually no change within the pH range 5.0 to 8.0 (Tables 8 and 9).

TABLE 7
Stability of εN-mPEG₁₀₀₀₀-αN-Laurate-Lysine-Phenylalanine¹

| | | | | | Rate |
|---|---|---|---|---|---|
| Buffer pH | Initial | Week 1 | Week 2 | Week 3 | (%/day) |
| | (%) | | | | |
| 5 | 100 | 98.8 | 79.9 | 14.2 | 2.51 |
| 7 | 100 | 72.2 | 40.5 | 4.7 | 2.18 |
| 8 | 100 | 63.9 | 40.5 | 26.5 | 2.84 |

¹calculated based on the concentration of αN-Laurate-Lysine-henylalanine(LLP)released:
$\% Conjugate = \frac{0.5 mM - {conc}^{LLC}}{0.5 mM} \times 100$

TABLE 8
Concentration of Lauric acid in the Stability Sample

| | | | |
|---|---|---|---|
| Initial | Week 1 | Week 2 | Week 3 |

| | |
|---|---|
| Buffer pH | (%) |

| | | | | |
|---|---|---|---|---|
| 5 | 0.50 | 0.47 | 0.53 | 0.55 |
| 7 | 0.50 | 0.46 | 0.52 | 0.54 |
| 8 | 0.50 | 0.44 | 0.53 | 0.59 |

TABLE 9
Concentration of Phenylalanine in the Stability Sample

| | | | |
|---|---|---|---|
| Initial | Week 1 | Week 2 | Week 3 |

| | |
|---|---|
| Buffer pH | (%) |

| | | | | |
|---|---|---|---|---|
| 5 | 0.50 | 0.50 | 0.51 | 0.54 |
| 7 | 0.50 | 0.50 | 0.53 | 0.60 |
| 8 | 0.50 | 0.50 | 0.48 | 0.58 |

The present invention provides for a composition that includes a protein such as alpha interferon or peptide such as insulin covalently conjugated to a substantially non-antigenic polymer, such as an alkyl or fatty acid terminated polyethylene glycol, via a linear spacer such as lysine or aminocarboxylic acid at an amino acid residue on the protein regardless the binding site, so as to provide the above-described properties which can be employed for pharmaceutical applications. Whenever the conjugate is coupling through a space, the length of the linker molecule is preferable less than 30 Å. Most preferable is between 8 to 15 Å.

What is claimed is:

1. A conjugate of a therapeutic protein, the conjugate comprising:
a therapeutic protein;
a carrier group including a non-antigenic hydrophilic polymer;
a linker disposed between the protein and the carrier group, said linker attached to the therapeutic protein via a carbamate bond and located directly adjacent to the carbamate bond, and said linker comprising a linear and saturated chain of between four and ten atoms of C and/or S.

2. The conjugate of claim 1, where the carrier group comprises a single liner polyethyleneglycol (PEG) chain.

3. The conjugate of claim 1, where the carrier group comprises a branched polyethyleneglycol (PEG) chain.

4. The conjugate of claim 1, where the carrier group comprises a PEG chain conjugated to a lipid or fatty acid.

5. A conjugate of a therapeutic protein, the conjugate represented by the formula: 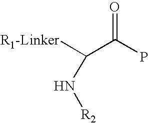
where P is a therapeutic protein;
where R1 is selected from the group comprising a non-antigenic hydrophilic polymer, a lipid or a fatty acid;
where R2 is selected from the group comprising a non-antigenic hydrophilic polymer, a lipid, a fatty acid, or two hydrogen atoms; and
a the linker comprises a linear and saturated chain of between four and ten atoms of C and/or S.

6. The conjugate of claim 5, where R1 and R2 comprise two polyethyleneglycol (PEG) chains.

7. The conjugate of claim 5, where R1 and R2 comprise a polyethyleneglycol (PEG) chain and a lipid moiety.

8. The conjugate of claim 1, where the linker is derived from lysine.

9. A method for preparing a conjugate of a therapeutic protein, the method comprising:
(step 1) conjugating one or more carrier groups to a linker, where the carrier groups are selected from the group comprising a non-antigenic hydrophilic polymer, a lipid or a fatty acid, and where the linker is a linear and saturated chain of between four and ten atoms of C and/or S; and
(step 2) conjugating the product of step 1 to a therapeutic protein or peptide via a carbamate bond.

10. A method of treating a patient with a therapeutic protein, where the therapeutic protein is formulated as a conjugate according to claim 1 or 5.
